(19) United States
(12) Patent Application Publication
(10) Pub. No.: US 2006/0019235 A1
(43) Pub. Date: Jan. 26, 2006

(54) Molecular and functional profiling using a cellular microarray

(75) Inventors: Yoav Soen, Palo Alto, CA (US); Daniel Shin-Yu Chen, Burlingame, CA (US); Patrick O. Brown, Palo Alto, CA (US); Mark Davis, Atherton, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/143,339

(22) Filed: Jun. 1, 2005

Related U.S. Application Data
(63) Continuation-in-part of application No. 10/190,425, filed on Jul. 2, 2002.
(60) Provisional application No. 60/303,109, filed on Jul. 2, 2001.
(60) Provisional application No. 60/577,159, filed on Jun. 4, 2004.

(51) Int. Cl. C12Q 1/00

(52) U.S. Cl. 435/4

(57) ABSTRACT

Cells are profiled with respect to their expression of cell surface molecules, and ability to respond to external stimulus in the microenvironment. External stimuli include cell-cell interactions, response to factors, and the like. The cells are arrayed on a planar or three-dimensional substrate through binding to immobilized or partially diffused probes. Probes of interest include specific binding partners for cell surface molecules, signaling cues that act to regulate cell responses, differentiation factors, etc., which may be arrayed as one or a combination of molecules.

This invention was made with Government support under contract HG009803 awarded by the National Institutes of Health. The Government has certain rights in this invention.

Living cells are defined by their elaborate patterns of protein expression, which control their persistence and behavior. These unique and elaborate sets of proteins provide for signaling pathways, interactions with other cells, structural variation, replication, metabolism, function, and the like. These proteins include cell surface molecules, which allow cells to probe their environment, and to exchange messages with their cellular and extracellular microenvironment. The behavior and fate of a cell is strongly dependent both on the internal state, and on complex cell-cell, cell-signal, and cell-ECM interactions mediated by such cell surface molecules.

Cellular signaling pathways, and the molecular components of these pathways, coordinate activities such as tissue growth, stasis, death and repair. Furthermore, a cell's interaction with its environment, including modification of the local environment to communicate with distant cells, is mediated by many secreted factors that directly or indirectly perform these tasks. Together, these patterns of signaling and response can provide a molecular and functional profile for a cell that dictates the cell's identity, role and behavior.

Cellular behavior can be defined by how a cell interacts with its environment, what functions it performs, what effectors it releases into its environment and what signals it provides to other cells. In order to understand the specific actions and capabilities of a cell, it is desirable to characterize the many factors a cell can produce in a given environment. The development of assays that can provide better, faster and more efficient prediction of cell behavior, cellular effects and clinical performance is of great interest in a number of fields, including clinical medicine where it can impact upon diagnosis, prognosis and treatment options for disease states such as cancer, autoimmunity, infectious disease and heart disease.

In addition to cellular phenotyping and characterization, there is substantial interest in methods of screening potential new targets and chemical entities for their effectiveness in physiologically relevant situations. Although the rewards for identification of a useful drug are enormous, but percentage of hits from any screening problem are generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound. The development of screening assays that can provide better, faster and more efficient prediction of mechanisms of action, cellular effects and clinical performance is of great interest in a number of fields, and is addressed in the present invention.

The ability to perform molecular and functional profiling of cells, including assessment of different cell types; and to assess and control cell fate/behavior; using automated high throughput data acquisition and advanced data analysis are of great interest for diagnostic, therapeutic, and research purposes.

### RELATED PUBLICATIONS

A protein microarray is described in International Patent Application WO00/63701. U.S. Pat. No. 4,591,570 discloses a matrix of antibody coated spots for determination of antigens. U.S. Pat. No. 5,858,801 (Brizzolara et al.) describes methods of patterning antibodies on a surface. International application WO02/12893 describes microarrays of functional biomolecules.

Immunophenotyping of cells using an antibody microarray is discussed in Belov et al. (2001) Cancer Research 61:4483-4489; in U.S. Pat. No. 5,866,350 (Canavaggio et al.); and U.S. Pat. No. 4,829,010 (Chang). International application WO02/39120 describes the use of antibody microarrays to identify the proteome of a cell.

Microarrays of cells expressing defined cDNAs are discussed in Ziauddin et al. (2001) Nature 411:107-110.

Cellular microarrays are described in U.S. Patent application 20030044389; and in U.S. Pat. No. 6,103,479 (Taylor). International application WO03/102578 describes methods of screening cellular responses using cellular components, test compounds and detector molecules in an array configuration. U.S. Pat. No. 6,573,039 discloses an optical system for intracellular profiling of cells using fluorescent reporter molecules.

### SUMMARY OF THE INVENTION

Compositions and methods are provided for molecular and functional profiling of homogeneous or heterogeneous populations of cells, in which cells are profiled with respect to their expression of cell surface molecules and secreted factors, their intracellular states, and ability to respond to external stimulus in the microenvironment. External stimuli include cell-cell interactions, response to factors, and the like. The cells are arrayed on a substrate through binding to immobilized or partially diffused probes, cells or fragments thereof. Cell immobilization on the array is based upon molecular recognition or adherence.

The use of a variety of surfaces and printing methods is also provided. In one embodiment of the invention, the substrate for the array is a hydrated, deformable hydrogel. Included are polyacrylamide hydrogels, preferably comprising components that weakly repulse cells, thereby providing low background binding. In one embodiment, the substrate comprises a polymerized mixture including acrylamide, and hydrophilic acrylates. In one embodiment of the invention, probes are printed on the substrate with a non-contact printer.

Probes of interest for use in the methods may be classified according to their function, which function can include the specific capture of cells (capture probes); the elicitation of a cellular response (effector probes); and the detection of molecules associated with a cell (detector probes). Probes, particularly capture probes, may be provided in a defined, specific geographic location, e.g. in an array format, and may be covalently bound to a substrate, non-covalently bound to a substrate, or partially diffused with respect to a substrate location. Probes may also be provided in a soluble form, particularly for the marking or detection of cells, cell products and metabolites, and the like. A variety of molecules find use as probes, including polypeptides, polynucleotides, polysaccharides, lipids; etc., and also including drug candidates, small detector molecules, and the like.

The methods of the invention allow for passive and active profiling of cells, including the characterization of cells by state, cell-surface marker, functional markers, etc. In functional profiling methods, parallel, programmed patterning of specific cell types and/or high-throughput stimulation of cells by a variety of immobilized or diffused cues, may be followed by phenotype examination and/or screening, and studies of cell-cell and cell-ECM interactions.

The ability to specifically capture cells onto defined locations at resolutions and feature sizes that are close to cellular dimensions allows for programmed cell patterning and enables close juxtaposition of different cell types, so that their mutual interaction can be examined. These features make the cell microarrays suitable for studying cell-cell and cell-ECM interactions, and for cell migration assays, secretion assays, and active and passive profiling assays. The microarray can optionally be incorporated into a multi-well-based platform by creating arrays within wells (intra-well printing).

### BRIEF DESCRIPTION OF THE DRAWINGS

FIG. 1. Co-spotting. Cells were specifically captured by capture probes in specific geographic regions. Secreted factors from the captured cells were assessed by co-spotted detector probes that captured the factors secreted by the cells.

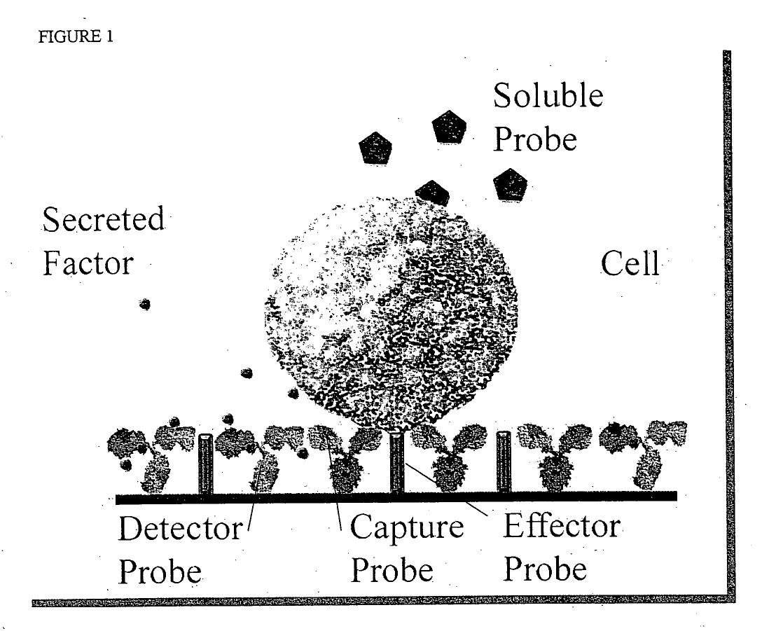

FIG. 2. Microscopic analysis. Captured cells were counterstained and/or specifically stained prior to visualization by light microscopy, fluorescent microscopy or electron microscopy.

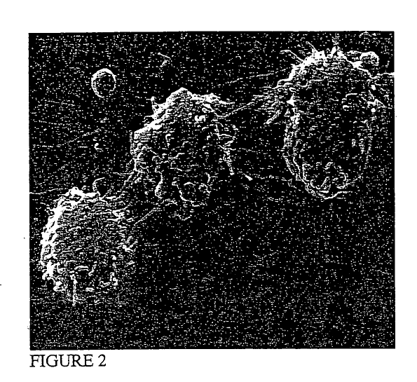

FIG. 3. Cells were captured by a capture probe (gp100/A2) and measured for secretion of specific factors by a detector probe (anti-IFN γ). A soluble probe (IL-2 or IL-15) was added to the cells, and its effect was measured. Exposure to IL-15, as opposed to IL-2, leads to greater responsiveness of T cells by IFN γ secretion.

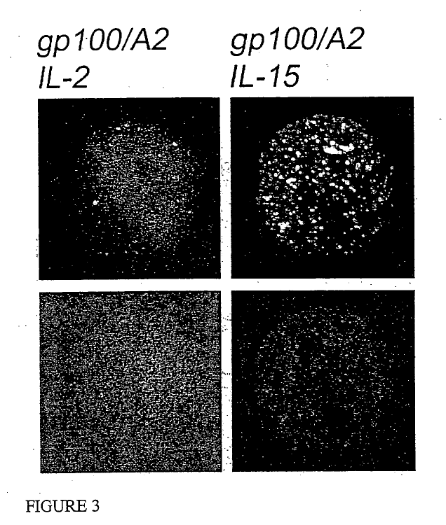

FIG. 4. Cells captured by capture probes (anti-CD3/anti-CD28) were measured for secretion of specific factors by a detector probe (anti-IFN γ). The addition of IL-2 as an effector probe on the right panel spots led to an amplified IFN γ secretion.

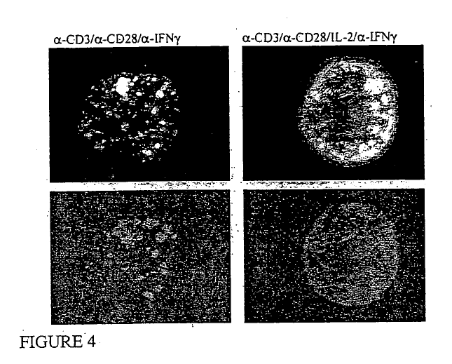

FIG. 5. Functional profiling of the immune response. CD8+ lymphocytes specific for a melanoma associated antigen MART-1 were specifically immobilized on the cellular microarray after recognizing their target. After recognition, they were activated and secreted factors detected by the cellular microarray. Secretion of interferon gamma, tumor necrosis factor alpha, granzyme B, GM-CSF and IL-2 were detected.

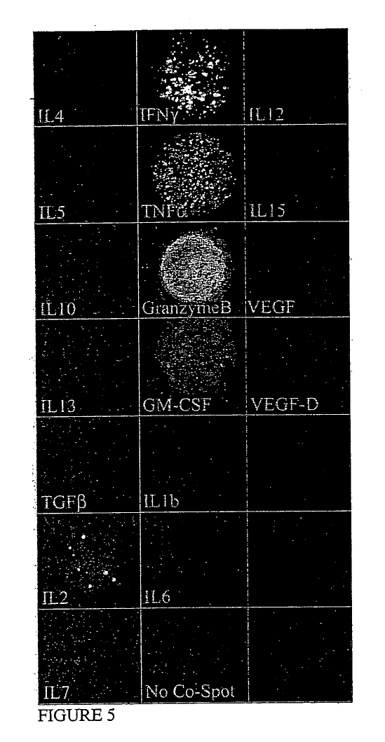

FIG. 6. Profiling of a solid tumor. Shown are three spots from a cellular microarray after application of malignant melanoma cells. A melanoma tumor sample was digested with collagenase and mechanically dissociated prior to application on the array. After cells from the sample were captured on the array, unbound cells were washed off and the remaining cells were exposed to a fluorescently tagged deoxyglucose molecule (6NDBG). Large melanoma cells fluoresced red due to uptake of the deoxyglucose molecule. Normal T cells from the sample, captured on the anti-CD3 spot, fluoresced weakly. Melanoma cells were captured by several capture probes, including anti-Her3 and anti CD117. The increased glucose uptake of melanoma cells reflects differences in cell behavior and implies a worse prognosis.

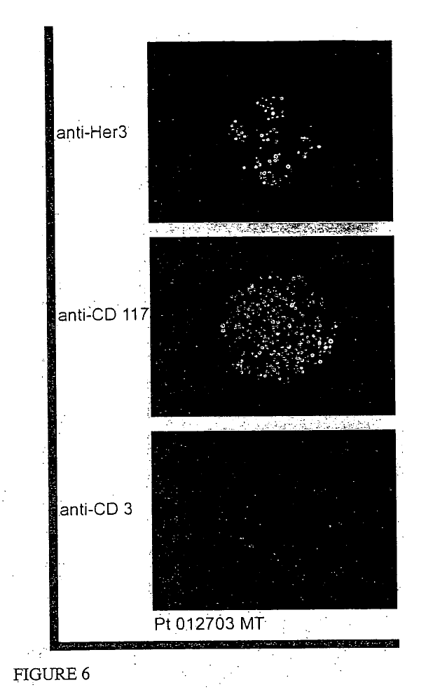

FIG. 7. Functional analysis. Cells specifically captured by a capture probe on the cellular array were loaded with the calcium sensitive dye Fura2, and calcium fluctuation was measured with single cell resolution.

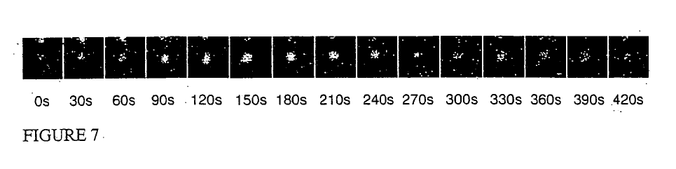

FIG. 8. Functional Analysis. A peptide-MHC specific CD8+ T cell was captured on the surface of the array by a specific capture probe. Based on recognition of its target, that cell captured a target tumor cell expressing the peptide-MHC recognized by the T cell and proceeded to kill it over a period of 20 minutes on the surface of the array.

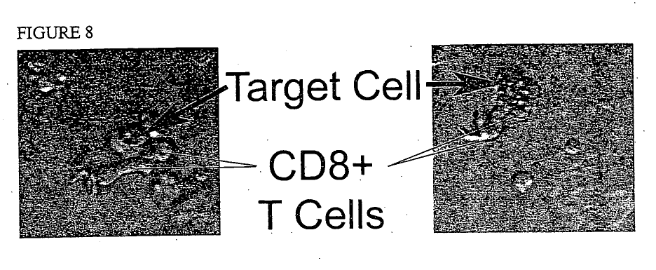

FIG. 9. Functional Analysis of cancer. A blood sample from a patient with leukemia was exposed to the surface of the array. The unbound cells were washed off and specifically bound cells remained adherent. Due to the tumor cells accounting for ˜90% of the cells in the sample, spots containing capture probes that recognize molecules on the surface of the leukemia cells were confluent, whereas spots containing capture probes that recognize molecules on the surface of normal cells, but not cancer cells were sparse. Some normal cells also express molecules that are on the leukemia cells, however, they account for a minority of the cells on those spots. The bound cells were exposed to C12-resazurin, which fluoresces in cells with increased reduction (vs. oxidation). The benign cells fluoresce, whereas the leukemia cells do not, reflecting the differences in functional state between the two cell types.

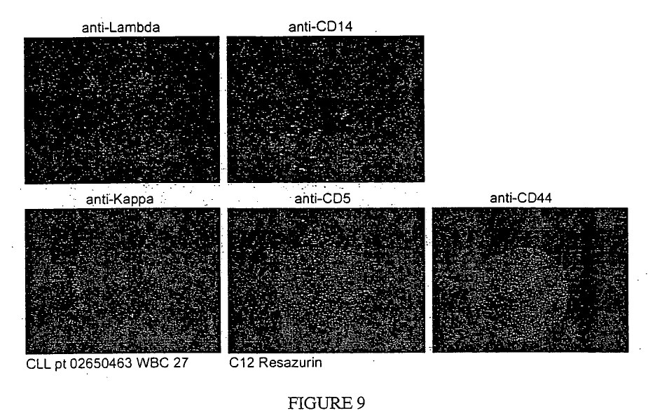

FIG. 10. Functional analysis. Interferon-gamma was detected by co-spotting of a capture probe and a detector probe (anti-interferon gamma). Spot number 4 from the left was co-spotted with 2 capture probes (anti CD3, anti-CD28), a detector probe (anti-Interferon-gamma, and an effector probe (rhlL-2) which increased the amount of interferon gamma secretion over anti-CD3, anti-CD28, anti-Interferon gamma spot alone (spot number 3 from the left).

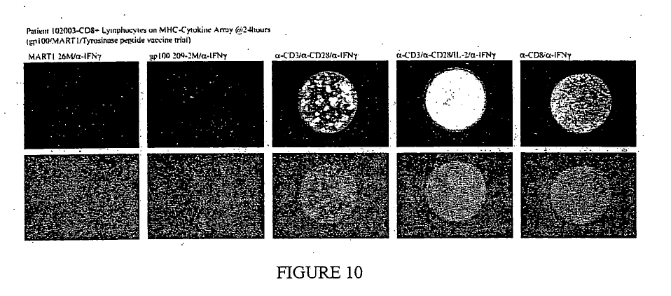

FIG. 11. Functional Profiling. Capture probes were mixed with detection probes and printed together on specific spots. Capture probes anti-CD20, anti-CD44 and anti-CD14 were mixed with 23 antibodies against secreted factors (only the anti-CD20 co-spots are shown). After development with a secondary antibody mixture, a pattern of secretion became obvious. The intermediate grade non-Hodgkin's lymphoma cells present in the clinical sample (ascites fluid) taken from a patient were captured by the anti-CD20. These cells were capable of secreting IL8 and TGF-beta, and to a lesser degree IL-4, IL13, MMP8, IL7 and CCL20, which is detected by a fluorescent signal on the surface of the array (not on the cell surface) reflecting secretion of these factors by specific lymphoma cells.

FIG. 12. Functional Profiling. High grade Non-Hodgkin's Lymphoma was analyzed for secretion of multiple factors. The cancer cells actively secreted multiple factors, including IL8, Angiogenin, and CCL17.

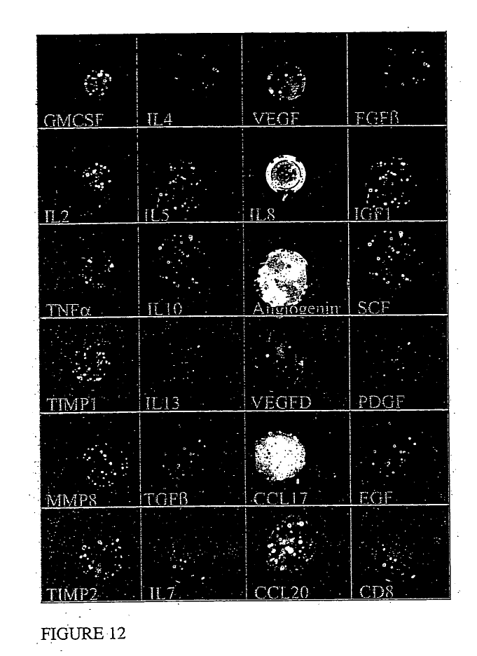

FIG. 13. Hypoxia Induced Functional Profiling. Colon cancer cells exposed to decreased oxygen (5% Oxygen in this example) showed increased secretion of Timp-2.

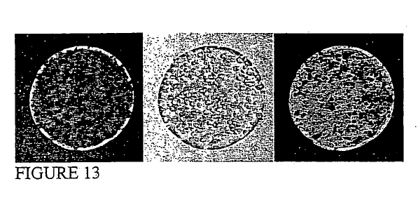

FIG. 14. Cellular response profiling to lipids. A preparation of peripheral blood monocytes (PBMC) from a normal control and from an acute coronary syndrome patient were profiled an arrays comprising, respectively, oxidized LDL, acetylated LDL, VLDL, HDL, ApoA, ApoB, ApoH, and CD8. It can be seen that the binding of cells to lipids associated with disease was increased in the sample from the acute coronary syndrome patient.

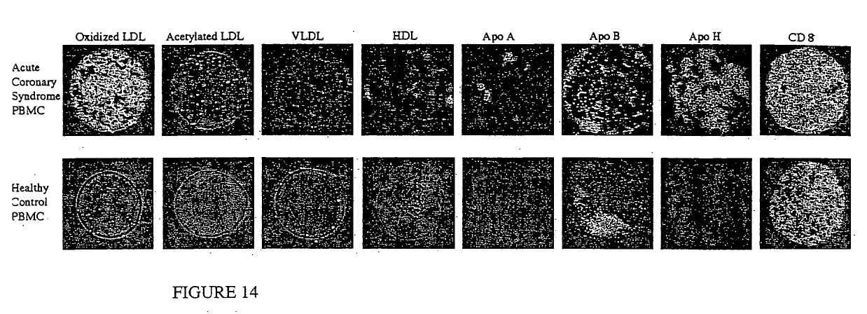

### DETAILED DESCRIPTION OF THE EMBODIMENTS

Cell profiling microarrays are used to characterize cells with respect to their expression of cell surface molecules, molecular interactions, behaviors and ability to respond to external stimuli in the microenvironment. External stimuli include cell-cell interactions, response to factors, cell interactions with their environment, and the like. The cells are arrayed on a substrate through binding to immobilized or partially diffused probes. After the cells are arrayed, they may be characterized, isolated or maintained in culture for a period of time sufficient to determine the response to a stimulus of interest. In one embodiment of the invention, the substrate for the array is a hydrated, deformable hydrogel, preferably comprising components that weakly repulse cells, e.g. a polymerized mixture comprising acrylamide, and hydrophilic acrylates.

The methods of the invention find use in clinical diagnosis for the profiling and classification of cell samples, e.g. biopsy samples, blood samples, and the like. Advantages of the invention include a fast, simple and inexpensive method of phenotyping clinical samples.

By providing for a controlled selection and position of cells, the signals, microenvironments and conditions that provide for a specific molecular and functional profile, cellular state, developmental path, or activation pathway can be explored in a systematic rigorous manner, in specific cell types or in heterogeneous cell samples. Such pathways can include, for example, stimulation of cells by proteins, lipids, other environmental cues, direct cell to cell contact, and the like, and may also include two way communication between cells of interest.

Utilizing the ability of cells to respond to exogenous signals, the present invention provides a unique tool for cell manipulation, utilizing selective or wide spectrum capture of cells and probe-mediated cell manipulation. Cell differentiation can be directed or manipulated in specific ways, and drugs can be screened for desired phenotypes. In addition, the methods can be used to search for passive and active markers present on cells, e.g. stem cells, cancer cells, etc.

Cell-microarrays offer advantages over existing multi-well-based approaches for cell stimulation and drug discovery. A microarray format supports an open microenvironment, wherein cells are free to move and explore neighboring environments printed on surrounding spots. Combining an open microenvironment concept with smaller feature sizes makes the cell-microarray format the method of choice for specific cell patterning, and assaying local cell stimulation, migration, secretion, cell-cell and cell-ECM interactions.

Any of the principles described here, can also be applied to a multi-well format, or flow cytometry. For example, a capture probe and a detector probe and/or an effector probe can be used to coat the bottom of a 96 well plate. Such a plate may then be used to detect secreted molecules from cells that have been specifically immobilized by the capture probe. Another possibility includes the use of a lipid such as oxidized-LDL, as a capture probe, with or without detector or effector probes, used to coat a 96 well plate, or as a labeled staining reagent for flow cytometry

The arbitrary choice of printed cues allows for reconstruction of well-defined micro-environments that can mimic essential features exhibited by their in-vivo counterparts, thereby serving as simplified model systems for studying their interactions with cells. By controlling the dose of a printed signaling probe, activation and response curves for specific cell types can be mapped out, and the events following activation can be imaged. Systematic mixing of cues can reveal the synergistic structure of a specific process. Likewise, collecting data in parallel from a comprehensive set of defined, naturally occurring signaling cues can lead to a dramatic boost in our understanding of the “language” utilized by cells. Cells of interest include a wide variety of types, each involving a multitude of important processes. For example, immune cells activated by antigens, cytokines or other stimulus or that are homing to tissues of interest; developing neurons interacting with signaling molecules, glia cells, or with vascular cells; embryonic stem (ES) cells progressing through early developmental pathways following fertilization; migrating and differentiating stem cells and cancer cells; cancer cells pulled out of their cell cycle, induced to commit apoptosis etc.

### DEFINITIONS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Substrate. As used herein the term “substrate” refers to any surface to which the probes are arrayed in defined, specific geographic locations. The array may comprise a plurality of different probes, which are patterned in a pre-determined manner, including duplicates of single probe types and combinations of different probes in a given spot.

In one embodiment of the invention, the substrate for the cellular microarray provides a high binding capacity for the spotted probe; may allow for probe localization with negligible diffusion; has a very low background binding for cells, and may provide for weak repulsion of cells; and provides an environment that does not adversely affect cell behavior or expression. A hydrated substrate can be desirable, as cells tolerate manipulation better in such an environment, and printed probes are exposed to a less caustic environment, protecting against a change in the characteristics of each spotted probe.

In applications that require high specificity of binding, a preferred substrate for the array is a hydrated, deformable hydrogel. Included as substrates are polyacrylamide hydrogels, preferably comprising components that weakly repulse cells, thereby providing low background binding. Hydrophilic components find use for this purpose. In one embodiment, the substrate comprises a polymerized mixture including acrylamide, and hydrophilic acrylates, which may be referred to herein as a high specificity substrate, or high specificity hydrogel.

Such high specificity substrates may be characterized in terms on non-specific cell binding, e.g. binding of cells to the substrate in the absence of a capture probe; binding of cells that are not reactive with a capture probe, and the like. Such non-specific binding is usually less than about 100 cells/mm², more usually less than about 10/mm², and may be less than about 1/mm². Those of skill in the art will understand that cells vary in their ability to adhere to a substrate; for example the non-specific binding of macrophages and monocytes may be much greater than the non-specific binding of lymphocytes. In general, adherent cells will tend to higher background “stickiness” than non-adherent cells.

The high specificity hydrogel substrate provides for hydration to bound cells and probes, high probe loading capacity, lack of diffusion of bound probes, low background binding of cells and free flow of cells across the surface of the microarray due to weak cell repulsion. Cells immobilized by spotted probe on this surface can continue to function in a physiologic manner, secreting factors and spreading out as visualized by electron microscopy.

A variety of other solid supports or substrates find use in the methods of the invention, including both deformable and rigid substrates. By deformable is meant that the support is capable of being damaged by contact with a rigid instrument. Examples of deformable solid supports include hydogels, polyacrylamide, nylon, nitrocellulose, polypropylene, polyester films, such as polyethylene terephthalate, etc. Also included are gels, microfabricated or bioengineered surfaces, microchannels, microfluidics, chambers, and patterned surfaces, which allow cells to reside in a three-dimensional environment, while still being completely or partially exposed to potentially immobilized or diffused probes (hydrogels, collagen gels, matrigels, ECM gels, etc). Herein, we refer to such realization as a 3D-array. Rigid supports do not readily bend, and include glass, fused silica, nanowires, quartz, plastics, e.g. polytetrafluoroethylene, polypropylene, polystyrene, polycarbonate, and blends thereof, and the like; metals, e.g. gold, platinum, silver, and the like; etc.

In addition, a rigid or deformable support may also incorporate a multi-electrode-array for electrical recording and stimulation or any other construct of interest onto which cues could be dispensed. Such a support may also incorporate the means to generate an electrical, magnetic field which may allow the cells to be repulsed from or attracted to the surface of the array, or agitated to increase individual cells to more regions or provide shear for adherent cells. Surfaces may also present biochemical attachment sites to immobilize and/or orient spotted probes.

Derivitized and coated slides are commercially available, or may be produced using conventional methods. For example, SuperAldehyde™ substrates contain primary aldehyde groups attached covalently to a glass surface. Coated-slides include films of nitrocellulose (FastSlides™, Schleicher & Schuelq, positively-charged nylon membranes (CastSlides™, Schleicher & Schuell), hydrogel matrix (HydroGel™, Packard Bioscience, CodeLink, Amersham), and simulated biologic surfaces (SurfaceLogix) etc.

The substrates can take a variety of configurations, including filters, fibers, membranes, beads, blood collection devices, particles, dipsticks, sheets, rods, capillaries, etc., usually a planar or planar three-dimensional geometry is preferred. The materials from which the substrate can be fabricated should ideally exhibit a low level of non-specific binding during binding events, except for methods where wide spectrum binding is preferred. Also, for functional profiling and manipulation experiments, the substrate should preferably be compatible with prolonged cell attachment and culturing.

In one embodiment of the invention, the substrate comprises a planar surface, and the binding members are spotted on the surface in an array. The binding member spots on the substrate can be any convenient shape, but will often be circular, elliptoid, oval or some other analogously curved shape. The spots can be arranged in any convenient pattern across or over the surface of the support, such as in rows and columns so as to form a grid, in a circular pattern, and the like, where generally the pattern of spots will be present in the form of a grid across the surface of the solid support. In some applications, labeled-probes are attached on and/or embedded in a substrate in a random order and their individual positions are inferred by analyzing their labels.

Array Preparation. The subject substrates can be prepared using any convenient means. One means of preparing the supports is to synthesize and/or purify probes, and then deposit the probes as a spot on the support surface. Probes can be prepared using any convenient methodology, such as automated solid phase synthesis protocols, monoclonal antibody culture, isolation from serum, lipid synthesis, protein folding reactions, carbohydrate purification, recombinant protein technology and like, using such techniques as are known in the art. The probes are spotted on the support using any convenient methodology, including manual techniques, e.g. by micro pipette, ink jet, pins, etc., and automated protocols.

In one embodiment, an automated spotting device is utilized, e.g. Perkin Elmer BioChip Arrayer™. A number of contact and non-contact microarray printers are available and may be used to print the binding members on a substrate. For example, non-contact printers are available from Perkin Elmer (BioChip Arrayer™), Labcyte and IMTEK (TopSpot™). These devices utilize various approaches to non-contact spotting, including piezo electric dispension; touchless acoustic transfer; en bloc printing from multiple microchannels; and the like. Other approaches include ink jet-based printing and microfluidic platforms. Contact printers are commercially available from TeleChem International (Arraylt™). Non-contact printers are of particular interest because they are more compatible with soft/flexible surfaces and they allow for a simpler control over spot size via multiple dispensing onto the same location.

Non-contact printing is preferred for the production of high-specificity cellular microarrays. With a non-contact printer, no solid printer part contacts the array surface. By utilizing a printer that does not physically contact the surface of substrate, no aberrations or deformities are introduced onto the substrate surface, thereby preventing uneven or aberrant cellular capture at the site of the spotted probe. Such printing methods find particular use with high specificity hydrogel substrates.

Printing methods of interest, including those utilizing acoustic or other touchless transfer, also provide benefits of avoiding clogging of the printer aperature, e.g. where probe solutions have high viscosity, concentration and/or tackiness. Touchless transfer printing also relieves the deadspace inherent to many systems, allowing the microzation of the probes themselves. The use of low shear forces, e.g. with acoustic transfer, also minimizes probe damage. To implement high-throughput printing, the use of print heads with multiple ports is preferred, and the capacity for flexible adjustment of spot size.

The total number of binding member spots on the substrate will vary depending on the number of different binding probes and conditions to be explored, as well as the number of control spots, calibrating spots and the like, as may be desired. Generally, the pattern present on the surface of the support will comprise at least about 2 distinct spots, usually at least about 10 distinct spots, and more usually at least about 100 distinct spots, where the number of spots can be as high as 50,000 or higher, but will usually not exceed about 10,000 distinct spots, and more usually will not exceed about 5,000 distinct spots. Each distinct probe composition may be present in duplicate or more (usually, at least 3 replicas) to provide an internal correlation of results. Also, for some tasks (such as stem cell fate manipulation and other cases, in which a group of cells tend to grow and occupy several spots) it is desirable to replicate blocks, each of several identical spots. In such cases replicate spots may be positioned in different neighboring spots to allow for estimation and compensation for potential cross talk effects (e.g. via soluble factors that are differentially secreted from cells on some of the spots). The spot will usually have an overall circular dimension and the diameter will range from about 10 to 5,000 μm, usually from about 100 to 1000 μm and more usually from about 200 to 700 μm. The binding member will be present in the solution at a concentration of from about 0.0025 μg/ml to about 50 μg/ml, and may be diluted in series to determine binding curves, etc.

By printing onto the surfaces of (preferably flat surfaced) multi-well plates, one can combine the advantages of the array approach with those of the multi well approach. Since the separation between tips in standard microarrayers is compatible with both a 384 well and 96 well plate, one can simultaneously print each load in several wells. Printing into wells can be done using both contact and non-contact technology, where the latter is also compatible with non-flat multi-well plates. The surface of the wells in the multi-well plate may be functionalized and/or coated so as to make them more compatible with specific cell-array applications. Other geometries, such as capillaries and blood collection tubes are also possible as substrates. Surface materials can also include nanotubes, modified or coated to allow binding of a capture probe. Surfaces which otherwise are not repellent of cells enough to adequately reduce background binding may also be used in association with a repellent coating, or an electric or magnetic field which weakly repulses cells from the array surface.

Probes, except for soluble probes, may be arrayed at a range of concentrations. Spots may comprise one, two, three or more different probes, and may combine capture, effector and/or detector probes. The amount of capture probe present in each spot is sufficient to provide for adequate binding of cells during the assay in which the array is employed.

A dilution series of a capture probe of interest will provide information regarding avidity of the interaction between the probe and its target on the cells. When the affinity of the interaction is known, the binding to a dilution series can be used to obtain an absolute measure for the expression level of the probe target. Alternatively, a relative measure of the expression levels can be obtained without the need for additional kinetic information by using a differential profiling experiment where two or more, differentially labeled cell populations compete on the binding to the same spots.

Within certain ranges of cells and binding members, the number of captured cells will be proportional to the expression level of the cognate protein, the affinity of the interaction, and the number of cells in the population capable of being captured and the exposure rate of cells to a particular geographic region. A dilution series may be used in the isolation of cells based on the expression level of the ligand for the capture probe. Cells expressing higher levels of the ligand will bind to spots comprising lower levels of capture probe. Spots with lower levels of capture probe can be used to enrich for cells expressing higher levels of cell surface target.

A dilution series can also be used for studying binding curves and/or phenotypic studies of cells that are sub-fractionated by the spots and/or for studying dose-dependent effects of effector probes, etc.

Differential pre-labeling of different cell populations followed by co-incubation on the slide and multi-color imaging facilitates discrimination of cells based on differences in expression of cell-surface markers, characterization of molecular markers that are differentially expressed on the cells, and identification and characterization of functional differences between the different cell types. In addition, the differential binding approach allows the usage of a common cellular reference that facilitates comparisons between different experiments and may be used for efficient screening of abnormal samples (e.g. by using a collection of normal samples as a reference).

The printing of probes, by which it is intended that a probe molecule is placed on the solid substrate in a specific location and amount, may be used to direct patterned assembly, migration, and programming of multicellular structures. For example, two distinct cell types may be juxtaposed in a specific physical orientation so that their interactions can be systematically observed.

### Probes

Probes used in the invention include capture probes, which are generally localized on the substrate; and effector probes and detector probes, which may be localized on the substrate or may be provided in soluble form before, during, and/or after the cells are applied to the array. Probes may be labeled with standard method known in the art including fluorophores, bead- or quantum-dot-conjugates. Distinct detection probes may be applied sequentially to the sample and/or pre-mixed prior to application. It will be understood by those of skill in the art that a soluble probe may also act as a capture, effector, or detector probe if it is to become immobilized on the array substrate after its application.

Capture Probe. Capture probes are specific binding partners for a cell surface molecule, used to capture a particular cell either by itself, or in combination with other capture probes. A member of a binding pair, i.e. two molecules, usually two different molecules, is one of the molecules (i.e., first binding member) that through chemical or physical means specifically binds to the other molecule (i.e., second binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used. In addition, in some cases a library of known or unknown compounds may be used to screen for binding partners and/or for stimulation effects upon binding.

Specific binding pairs of interest include carbohydrates and lectins; complementary nucleotide sequences; peptide ligands and receptors; effector and receptor molecules; hormones and hormone binding protein; enzyme cofactors and enzymes; enzyme inhibitors and enzymes; peptides, proteins, protein containing molecules, cytokines and growth factors, peptide-MHC complexes, supernatant from cell cultures; extracellular matrix components; cell adhesion molecules; target cells, and extracts from specific cells; microbes, drugs, lipids, lipoproteins and their receptors; antibodies, antibody fragments, immunoglobulins, and peptide/MHC complexes; complement system components; chemical modifications of ligands, proteins, lipids and lipoproteins; small molecules and chemical compounds, etc.

The specific binding pairs may include analogs, derivatives and fragments of the original specific binding member. For example, a receptor and ligand pair may include peptide fragments, chemically synthesized peptidomimetics, labeled protein, derivatized protein, etc.

Specific capture probes of interest include antibodies and fragments thereof, which may bind, for example, cell surface antigens; adhesion molecules; extracellular matrix components; receptor ligands; antigen-bearing MHC constructs; lipids; therapeutic agents; polyproteins; microbial components; complex cell constituent, e.g. cell membranes; cell extract and the like; including complete cells, which may be live or fixed carbohydrates and carbohydrate-containing molecules, lectins, etc. The affinity and specificity of the binding members lead to a unique cell attachment pattern reflecting the levels of expression of surface antigens. Polypeptide, glycoproteins, proteoglycans, and lipoprotein binding probes are of particular interest, including those found in extracellular matrix and body fluids.

Probes that are specific binding partners for many different cell types provide an adherent surface for one or more cell types may be referred to as wide spectrum probes, and find use in methods for less selective capture, which methods are optionally combined with the use of selective effector and/or detector probes.

In another embodiment, specific capture, and/or detector, and/or effector probes are randomly scattered and subsequently identified using encoded tags, e.g. color-coding, nano-particle attachments, specific chemical modifications, DNA sequence tags, molecular beacons, specific protein tags, micro-transponders and the like. Examples include probe-coated beads, probe-coated quantum dot conjugates, membrane-bound vesicles that may display specific probes on their membranes and may carry diffusible factors, biodegradable polymer beads for fast or gradual release of effector molecules, and the like. These probes may be attached to a surface, embedded in a gel-like layer, and/or applied in solution to immobilized cells, cells embedded in a gel-like layer, and/or to immobilized factors that were secreted by the cells.

Capture probes of interest include, without limitation, antibodies specific for: CD1A; CD1B; CD1C; CD1D; CD3; CD4; CD5; CD6; CD7; CD8; CD9; CD10; CD11a; CD11b; CD11c; CD13; CD14; CD15S; CD19; CD20; CD22; CD23; CD25; CD26; CD30; CD31, CD33; CD34; CD35; CD36; CD38; CD39; CD40; CD44; CD45; CD46; CD47; CD55; CD57; CD59; CD60B; CD135; CD144; CD56; CD106; CD54; CD107A; CD107B; CD66b; CD66f; CD69; CD73; CD105; CD29; CD18; CD61; CD49a; CD49b; CD49c; CD49d; CD49e; CD49f; CD11a/LFA-1; CD11b; CD11c; CD51-61; CD103; CD104; CD41A; CD41b; CD42a; CD42b; CD44; CD62e; CD62L; CD62p; CD66b; CD68; CD70; CD71; CD72; CD80; CD81; CD83; CD84; CD86; CD87; CD88; CD94; CD90; CD100; CD109; CD110; CD114; CD116; CD117; CD120a; CD120b; CD121a/SIL-1RI; CD122; CD127; CD130; CD134; CD138; CD140a; CD140b; CD141; CD147; CD150; CD151; CD152; CD153/CD30L; CD154; CD162; CD165; CD166; CD180; CD183; CD150; CD151; CD152; CD153/CD30L; CD154; CD162; CD165; CD166; CD180; CD183; CD184; CD195; CD200; CD212; CD223; CD221; CD220; CD206; CD137; CD21; CD22; CD172a/b; CD172b; CD222; CD231; CD8 FITC; CD15; CD16; CD19; CD20; CD27; CD30; CD37; CD43; CD45RO; CD45RA; CD48; CD50; CD63; CD64; CD66d; CD74; CD77; CD91; CD92; CD97; CD98; CD99; CD99R; CD101; CD137; CD146; CD158a; CD158b; CD160; CD161; CD164; CD201; CD206; CD209; CD220; CD226; CD227; CD229; CD235a; CD244; etc.

Also of interest are included p-Cadherin; Cadherin-5; Beta7 integrin; PRR2; FMS; IFN-gamma Ralpha; IL-4 Ralpha; CDW125; IL-6 R; CDW128; CDW128b; CDW210; CCR6; FMLP R; P-GP; MUC2; HLA-ABC; Galectin-3; GP230; MU-Calpain; APEP A; LMP-1; Siglec-6; TAP2; Thymus Medulla; CDW93/C1QRP; α-human Activin RIA; α-human Activin RIB; α-human Activin RIIA/B; α-human Activin RIIB; α-human ALCAM; α-human ALK-1; α-human AxI; α-human BAFF; α-human BMPR-IB/ALK-6; α-human BMPR-II; α-human CNTF Rα; α-human Contactin-1; α-human DR6/TNFRF21 (Death recptor 6); α-human Dtk; α-human Ephrin-A3; α-human Ephrin-A4; α-human Ephrin-B3; α-human ErbB3; α-human Frizzled-3; α-human Frizzled-7; α-human GFRα-3 (GDNF receptor α3); α-human gp130; α-human HGF receptor; α-human Leptin R; α-human MCAM; α-human MER; α-human MSP receptor; α-human NCAM-L1; α-human Neuritin; α-human SCF receptor; α-human Semaphorin 6A; α-human Tie-1; α-human Tie-2; α-human TNF RI/TNFRSF1A; α-human TNF RII/TNFRSF1B; α-human TRAIL R2/DR5/TNFRSF10B; α-human TRAIL R3/DcR1/TNFRSF10C; α-human TRAIL R4/DcR2/TNFRSF10D; α-human TrkA Neurotrophin receptor; α-human TrkB Neurotrophin receptor; α-human TROP-2; α-human TSLP receptor; α-human uPAR; α-human VCAM-1; α-human VEGF R1 (Flt-1); α-human VEGF R3 (Flt-4);; α-human A2B5; α-human D6; α-human DAN; α-human EpCAM; α-human DR3/TNFRSF25; α-human Endoglycan/PODLX2; α-human CCR8; α-human ErbB4; α-human ErbB2; α-human FGF R1 (IIIb); α-human FGF R2; α-human FGF R3; α-human FGF R4; α-human VEGF R2 (KDR); α-human M-CSF R; α-human GHR (growth Hormone Receptor); α-human HVEM/TNFRSF14; α-human NRG-1-β1/HRG-β1; α-human Glucose Transporter Type 1 (Glut1); α-human Glucose Transporter Type 2 (Glut2); α-human Glucose Transporter Type 3 (Glut3); α-human Glucose Transporter Type 5 (Glut5); α-human GDNF R α-4 (GDNF receptor α4); α-human Nogo Receptor (NgR); α-human OX40 ligand; α-human Jagged-1; α-human Oligodendrocyte marker O1; α-human Oligodendrocyte marker O4; α-human Thrombopoietin receptor; and the like.

Lipids used as capture probes required individual reconstitution in different resuspension media to get adequate solubilization or resuspension. Otherwise, they were spotted in a similar fashion as other capture probes. Any lipid or lipid containing substance can be useful for analysis of cell responses to those substances. Of particular interest in heart disease, are compounds known to play a role in this disease, such as LDL, oxLDL, acLDL, HDL, VLDL, triglycerides, apoproteins, cholesterol. Cell samples of interest include whole blood, buffy coat preps, PBMCs, PBLs, monocytes, lymphocytes, neutrophils, and single cell suspensions of biopsies (such as an atheroma). Also of importance is co-spotting to measure functional responses to binding to these lipids and lipid-containing compounds.

Effector Probes. Effector probes are molecules that elicit a cellular response, e.g. by providing signaling cues that regulate cell responses, differentiation factors, effect cell survival or behavior, etc. Effector probe may also function as a capture probe, or may be provided in conjunction with a capture probe. Likewise, an effector probe may also be used as a detector probe. Effector probes that generate signals or affect the cell's growth, act to regulate cell responses, differentiation, migration, viability and apoptotic potential, gene expression, chromatin rigidity, morphological phenotypes and the like may be used. Effector probes may be bound to the microarray substrate, partially diffused on the substrate, and may also be soluble, and applied before, during or after binding of cells to the substrate.

Any molecule capable of eliciting a phenotypic change in a cell may be used as an effector probe. Effector probes may be the products of other cell types, e.g. expressed proteins associated with a disease, or secreted in a normal situation or during development; may be compounds associated with the ECM; may be naturally occurring factors, analogs or mimetics thereof; may be fragments of cells, may be surface membrane proteins free of the membrane or as part of microsomes, etc. Useful effector probes also include a variety of polypeptides, chemicals, therapeutic agents, lipids, carbohydrates and other biologically active molecules, e.g. chemokines, cytokines, growth factors, differentiation factors, drugs, polynucleotides, etc.

Effector probes may be used individually or in combination. Illustrative naturally occurring factors include cytokines, chemokines, differentiation factors, growth factors, soluble receptors, hormones, prostaglandins, steroids, drugs, oxidized LDL, etc., that may be isolated from natural sources or produced by recombinant technology or synthesis, compounds that mimic the action of other compounds or cell types, e.g. an antibody which acts like a factor or mimics a factor, such as synthetic drugs that act as ligands for target receptors. For example, in the case of the T cell receptor, the action of an oligopeptide processed from an antigen and presented by an antigen-presenting cell, etc. can be employed. Where a family of related factors are referred to with a single designation, e.g. IL-1, VEGF, IFN, etc., in referring to the single description, any one or some or all of the members of the group are intended. Compounds are found among biomolecules including peptides, saccharides, fatty acids, steroids, purines, pyrimidines, oligonucleotides, polynucleotides, derivatives, structural analogs or combinations thereof.

Effector probes can include cytokines, chemokines, and other factors, e.g. growth factors, such factors include GM-CSF, G-CSF, M-CSF, TGF, FGF, EGF, BMP, Shh, Wnt, TNF-α, GH, corticotropin, melanotropin, ACTH, etc., extracellular matrix components, surface membrane proteins, such as Notch and its ligands, integrins, cadherins, and adhesins, ephrins, semaphorins and their ligands, and other components that are expressed by the targeted cells or their surrounding milieu in vivo. Components may also include soluble or immobilized recombinant or purified receptors, or antibodies against receptors or ligand mimetics. Effector probes may be mixed in arbitrary combinations and gradients and may combined with capture and/or detection probes. Effector probes may include un-identified mixtures such as conditioned media and cellular supernatant and/or unknown components from a library of peptides, proteins, lipids, lipoproteins, hormones, vitamins, small molecules, DNA, RNA, drugs, etc

Included are pharmacologically active drugs, genetically active molecules, etc. Compounds of interest include chemotherapeutic agents, morphogenes, apoptotic agents, anti-inflammatory agents, hormones or hormone antagonists, ion channel modifiers, and neuroactive agents. Exemplary of compounds suitable as binding pair members for this invention are those described in The Pharmacological Basis of Therapeutics, Goodman and Gilman, McGraw-Hill, New York, N.Y., (1993) under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

As detectors, antibodies against the molecules may be used. As the molecule itself, they are effectors, including 4-1BB; Adiponectin/Acrp30; AgRP; ANG; Angiopoietin-2; AR; B7-H1; BDNF; BLC/BCA-1; BMP-4; BMP-6; BMP-7; BTC; CCL28/VIC; Ckb8-1; CNTF; CTACK; CXCL16; EGF; ENA-78; Eotaxin; Eotaxin-2; Eotaxin-3; FGF basic; FGF-4; FGF-6; FGF-7/KGF; FGF-9; Flt-3; Fractalkine; GCP-2; G-CSF; GDNF; GITR Ligand; GITR; GM-CSF; GROa; HCC-4; HGF; I-309; I-TAC; IGF-I; IGFBP-1; IGFBP-2; IGFBP-3; IGFBP-4; IGFBP-6; IL-1α; IL-1β; IL-1rα; IL-3; IL-6; IL-7; IL-8; IL-11; IL-12 p40; IL-12 p70; IL-13; IL-15; IL-16; IL17; IP-10; Leptin; LIGHT; Lymphotactin; M-CSF; MCP-1; MCP-2; MCP-3; MCP-4; MDC; MIF; MIG; MIP-1a; MIP-1b; MIP-1delta; MIP-3a; MIP-3b; MMP-8; MSP; NAP-2; beta-NGF; NT-3; NT-4; OPG; OSM; PARC; PDGF-BB; PIGF; RANTES; SCF; SDF-1a/b; SDF-1b; TARC; TECK; TGF-a; LAP TGF-β1; TGF-beta 2; TGF-beta 2; TIMP-1; TIMP-2; TNF-α; TNF-β; TPO; VEGF; VEGF R3; VEGF-D; and the like.

Detector Probes. Detector probes allow detection of a cell phenotype, response, expression product, etc. Detector probes may also function as a capture probe, or may be provided in conjunction with a capture probe; and may also function as, or in conjunction with, an effector probe. Likewise, an effector probe may also be used as a detector probe. Detector probes may be bound to the microarray substrate, partially diffused on the substrate, and may also be soluble, and applied before, during or after binding of cells to the substrate.

Detector probes of interest include a variety of polypeptides, chemicals, therapeutics, lipids, carbohydrates and other molecules that can interact with an antigen expressed on the cells, a factor secreted by a cells, or recognize an effect caused by a cell or cell secreted factor, e.g. monoclonal antibody against a secreted factor, reagents that fluoresce when oxidized by a cell or cell factor, molecular sensors of functional processes like metabolic activity, intracellular enzymatic activity, drug resistance, calcium fluxes etc. Binding of secreted factors to detection probes can be detected, in some cases, by development with a labeled secondary probe, or change in a physical property, as necessary. In addition, detector probes can function as a specific binding partner, or report a readout for a molecule or factor that is not attached to the cell surface, such as secreted or shed factors.

Detector probes of interest also include counterstaining with a monoclonal antibody or stain, labeled deoxyglucose to determine glucose metabolism, Rhodamine 123 staining to reflect mitochondrial potential, detection of cytokines that affect T cell survival and activation and secretion of other cytokines, etc.

Detector probes also include soluble probes that can interact with a molecule on the surface of the cellular microarray (the cells, the surface, other probes, the solution and its contents) that can be applied to the microarray or the cellular solution prior to, during, or after application of the cellular sample to the microarray. Soluble probes can mark different cell types, stain for different cell states, report biochemical pathways, or otherwise affect or mark the conditions on the microarray.

Cells. Cells for use in the assays of the invention can be an organism, a single cell type derived from an organism, or can be a mixture of cell types, as is typical of in vivo situations, but may be the different cells present in a specific environment, e.g. blood, vessel tissue, liver, spleen, heart muscle, brain tissue, malignant aspiration, biopsy, excision or resection, etc. Microbes can be utilized in a similar fashion as cells.

The invention is suitable for use with any cell type, including primary cells, prokaryotic and eukaryotic cells, adherent and suspension cells, normal and transformed cell lines, cells from transgenic animals, transduced cells, cells with reporter genes (and/or other biochemical reporters), and cultured cells, which can be single cell types or cell lines; or combinations thereof. In assays, cultured cells may maintain the ability to respond to stimuli that elicit a response in their naturally occurring counterparts. Cultured cells may have gone through up to five passages or more, sometimes 10 passages or more. These may be derived from all sources, particularly mammalian, and with respect to species, e.g., human, simian, rodent, etc., although other sources of cells may be of interest in some instances, such as bacteria, plant, fungus, viruses, prions, etc.; tissue origin, e.g. heart, lung, liver, brain, vascular, lymph node, spleen, pancreas, thyroid, esophageal, intestine, stomach, thymus, malignancy, atheroma, pathological lesion, etc.

In addition, cells that have been genetically altered, e.g. by transfection or transduction with recombinant genes or by antisense technology, to provide a gain or loss of genetic function, may be utilized with the invention. Methods for generating genetically modified cells are known in the art, see for example “Current Protocols in Molecular Biology”, Ausubel et al., eds, John Wiley & Sons, New York, N.Y., 2000. The genetic alteration may be a knock-out, usually where homologous recombination results in a deletion that knocks out expression of a targeted gene; or a knock-in, where a genetic sequence not normally present in the cell is stably introduced.

A variety of methods may be used in the present invention to achieve a knock-out, including site-specific recombination, expression of siRNA, anti-sense or dominant negative mutations, and the like. Knockouts have a partial or complete loss of function in one or both alleles of the endogenous gene in the case of gene targeting. Preferably expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence.

Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of the targeted genes. A functional knock-out may also be achieved by the introduction of an anti-sense construct that blocks expression of the native genes. “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration.

A genetic construct may be introduced into tissues or host cells by any number of routes, including calcium phosphate transfection, endocytosis, viral infection, microinjection, or fusion of vesicles. Jet injection may also be used for intramuscular administration, as described by Furth et al. (1992), Anal Biochem 205:365-368. The DNA may be coated onto gold microparticles, and-delivered intradermally by a particle bombardment device, or “gene gun” as described in the literature (see, for example, Tang et al. (1992), Nature 356:152-154), where gold microprojectiles are coated with the DNA, then bombarded into cells.

Cell types that can find use in the subject invention include stem and progenitor cells, e.g. embryonic stem cells, hematopoietic stem cells, mesenchymal stem cells, neural stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells, etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

These methods of the invention can be applied to both adherent, e.g. epithelial cells, endothelial cells, neural cells, etc., and non-adherent cells. After the cells are captured on the array, they may be characterized, or maintained in culture for a period of time sufficient to determine the response to a stimulus of interest. To examine specific cell-cell interactions, different cell populations may be co-captured by the same probe or, alternatively on adjacent probes. The irrelevant, unbound cells can then be removed by washing. Alternatively, one cell population can be captured and isolated on the array and subsequently used to capture another cell population that cannot be captured by the first probe. Cells may be removed from the surface of the array, e.g. by local aspiration or via global transfer to a different medium. A particularly important method for global transfer that can preserve the structure of the array is the transfer of array-bound, isolated cells into a gel matrix (or the like). A simple realization of this kind of transfer is achieved specific capture of cells onto an inert substrate (e.g. hydrogel and the like), followed by matrigel polymerization onto the cells (with or without additional factors that promote cellular migration), and further incubation period during which the cells can migrate into the gel layer. In most cases, the gel layer is more suitable for studying specifically-isolated cell clusters in 3d environment and in most cases will offer better conditions for expanding the cells. In addition, it may assist in specific cell removal by cutting pieces from the gel followed by standard cell extraction methods.

In order to profile adherent cells, it is often preferred to dissociate them from the substrate that they adhered to, and from other cells, in a manner that maintains their ability to recognize and bind to probe molecules. Methods of dissociating cells are known in the art, including protease digestion, etc. Preferably the dissociation methods use enzyme-free dissociation media or mild enzymatic dissociation. Alternatively, the cells may be dissociated enzymatically and left to recover prior to the interaction with the array. In some cases (e.g., those involving non-specific capture followed by functional profiling), the cells may be applied to the array immediately following enzymatic dissociation. Cells may be applied to the array either in suspension or within ECM gels, agar, etc. Dissociation of tissue into single-cell suspensions is appropriate prior to application to the array. Such dissociation includes physical dissociation and/or enzymatic dissociation with reagents such as collagenase, and is well described.

Microenvironment. The cellular microenvironment, or environment, encompasses cells, media, factors, time and temperature. Environments may also include drugs and other compounds, particular atmospheric conditions, pH, salt composition, minerals, etc. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such a fetal calf serum and/or conditioned media, or media which is fully defined and serum free. A variety of culture media are known in the art and commercially available. Typically, RPMI supplanted with 5% FCS, and 1× Penicillin/Streptomycin/Glutamine is used. However, phosphate buffered saline also works well if longer integrity of the cells is not required.

Phenotype. Various cellular outputs may be assessed to determine the response of the cells to the input variable, including calcium flux, BrdU incorporation, expression of molecular markers (e.g. differentiation markers), secretion of specific factors (e.g. MMPs, cytokines etc.), localization of specific factors, expression of an endogenous or a transgene reporter, metabolic reporters, intracellular chemical modifications (e.g. extent of specific chromatin methylations) electrical activity (e.g. via voltage-sensitive dyes), release of cellular products, cell motility, size, shape, viability and binding, etc. In some case (such as when cells are embedded in a 3D gel), even local pH levels or O₂ and CO₂ concentrations can be assayed. The phenotype may be examined in real time on live cells and/or at the end of the experiment (on live or fixed cells). Generally the analysis provides for site specific determination, i.e. the cells that are localized at a spot are analyzed for phenotype in an individual or spot specific manner, which correlates with the spot to which the cells are localized.

The phenotype of the cell in response to an effector probe or a microenvironment may be detected through changes in various cell aspects, usually through parameters that are quantifiable characteristics of cells. Characteristics may include cell morphology, growth, viability, metabolic activity, drug resistance activity, intracellular pH, expression of genes of interest (e.g. as viewed by the intensity of staining with a specific marker), presence and localization of proteins of interest, cell motility, change in secretion profile, interaction with other cells, and include changes in quantifiable parameters, parameters that can be accurately measured. The cellular phenotype may include one or more measured properties, collectively defining a composite phenotype. Data collected from the array (e.g. by manual and/or automatic acquisition of images followed by measurements of features revealed by the images) includes cell-based statistics (collection of composite phenotypes from individual cells), spot-based statistics (averages of composite phenotypes over the entire spot region), and compound-based statistics (averages over spots with the same composition). The measured statistics may be stored in a database and used for building phenotype profiles and knowledge bases that are characteristics of a disease, and/or correlate with recovery or recurrence. Multi-parameter phenotyping may also be used for examining similarities, differences, and interactions between substances.

A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. Parameters may provide a quantitative readout, in some instances a semi-quantitative or qualitative result. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, frequently biopolymers, e.g. polypeptides, polysaccharides, polynucleotides, lipids, etc. Cell surface and secreted molecules are a useful parameter type as these mediate cell communication and cell effector responses and can be readily assayed. In one embodiment, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide, e.g. a phosphorylated protein, such as a STAT transcriptional protein; or sulfated oligosaccharide, or such as the carbohydrate structure Sialyl Lewis x, a selectin ligand.

A parameter may be defined by a specific monoclonal antibody or a ligand or receptor binding determinant. Parameters may include the presence of cell surface molecules such as CD antigens (CD1-CD247), cell adhesion molecules including integrins, selectin ligands, such as CLA and Sialyl Lewis x, and extracellular matrix components. Parameters may also include the presence of secreted products such as lymphokines, chemokines, etc., including IL-2, IL-4, IL-6, growth factors, etc.

Data Acquisition. In implementations of cellular microarrays where high throughput molecular and functional profiling is desired, an appropriate method of high throughput data acquisition is required for enablement. Cell microarrays can be scanned to detect binding of the cells, e.g. by using a simple light microscopy, scanning laser microscope, by fluorimetry, a modified ELISA plate reader, etc. For example, a scanning laser microscope may perform a separate scan, using the appropriate excitation line, for each of the fluorophores used. The digital images generated from the scan are then combined for subsequent analysis. For any particular array element, the ratio of the fluorescent signal with one label is compared to the fluorescent signal from the other label DNA, and the relative abundance determined.

Generally, optical scanning is preferred, using an automated microscope and a motorized stage. Robotic loading of slides onto the microscopy platform allows a further increase in throughput. Cellular microarrays can be marked with predetermined geographic locations that allows identification of array start and stop points. This can be achieved using a spot containing a visible dye, a fluorescent dye or marker or an expected cell binding pattern at a particular location. In the simplest implementation, a single spot is thus labeled, marking a position on the array grid, such as in one corner. In more sophisticated implementations, all corners, or pre-determined patterns of markers are printed. Once these markers are identified, automated data acquisition in all involved channels may be performed (for example, but not limited to brightfield/phase contrast/DIC/Color, FITC, CY5, CY3, DAPI, PI, UV, etc.). Automated analysis is also of interest, allowing automated counting of cells binding to each spot, cell morphology, fluorescence intensity, etc. Automated analysis may include comparison with an established database, clustering by phenotype, etc.

### Profiling Methods

Passive Profiling. In methods of passive profiling, a suspension of cells, which may be adherent cells or non-adherent cells, is allowed to bind to a microarray of capture probes. The population of cells, as described above, is added to a microarray comprising bound probes. The suspension is applied to the substrate without a cover or under a coverslip, or into a fixed volume of “hybridization” or “staining” media; or in a “perfusion” chamber.

Suitable capture probes include any type of molecule capable of sufficiently strong and specific interaction with cells. In one embodiment, the probe is an antibody or fragment thereof. In another embodiment, the probe is a polypeptide other than an antibody, including cell adhesion molecules (CAMs), peptide-MHC (p-MHC) and extracellular matrix (ECM) components, e.g. laminin, fibronectin, collagen, vitronectin, tenascin, restrictin, hyaluronic acid, etc. cytokines; growth factors; and the like. Another embodiment, the probe is a lipid, lipid complex, or lipid containing complex or molecule such as cholesterol, LDL, oxLDL, acLDL, small dense LDL, HDL, IDL, VLDL, VLDL remnants, triglycerides, ApoA1, ApoB, ApoB-100, ApoH, Lp a1, Lp a2. In another embodiment, the probe is a carbohydrate, or carbohydrate containg complex or molecule. The incubation time should be sufficient for cells to bind the probes. Generally, from about 4 minutes to 1 hr is sufficient, usually 20 minutes sufficing. The incubation temperature varies between application, from 4 degrees C to 37 or 39 degrees C, or higher.

While many assays are performed with live cells, assays may also be performed with fixed cells. Cells fixed with various concentrations of reagents such as PFA, glutaraldehyde, methanol, acetic acid, etc. can be used alone, or in comparison with non-fixed cells.

After incubation, the insoluble support is generally washed to remove unbound and non-specifically bound cells in any medium that maintains the viability of the cells and the specificity of binding, e.g. RPMI, DMEM, Iscove's medium, PBS (with Ca⁺⁺ and Mg⁺⁺), etc. The number of washes may be determined experimentally for each application and cell type, e.g. by observing the degree of non-specific binding following each wash round. Usually from about one to six washes are sufficient, with sufficient volume to thoroughly wash non-specifically bound cells present in the sample.

Such profiles can be absolute or differential. In an absolute profile, a single cell type is added to the microarray, and the number of bound cells detected. Occupied spots denote the presence of the corresponding cell surface marker to the binding probe. Over a range of cell and probe concentrations, the higher the expression level, the higher the number of captured cells.

A differential profile is a competitive assay, where two or more cell types/populations are pre-labeled with different labels, combined and applied to a single slide, where they compete for binding to probe molecules. Following washout, the slide can be scanned and scored for the relative number of label present for each of the cell types.

In order to detect the presence of bound cells from each type, a variety of methods may be used. In an absolute assay, the cells need not be labeled at all or may be labeled with a detectable label, and the amount of bound label directly measured. In a differential assay, labeled cells may be mixed with differentially labeled, or unlabeled cells and the readout can be based either on the relative number of pixels with a given label (or no label, respectively) or the relative number of cells with a given label (or no label, respectively). In yet another embodiment, the cells themselves are not labeled, but cell-type-specific second stage labeled reagents are added in order to quantitate the relative number of cells from each type , or to phenotype the cells. In some instances the cells will not be quantitatively measure, but will be observed for such phenotypic variation as morphology, adherence, etc.

Examples of labels that permit direct measurement of bound cells include radiolabels, such as ³H or ¹²⁵I, fluorescers, dyes, beads, chemilumninescers, colloidal particles, and the like. Suitable fluorescent dyes are known in the art, including fluorescein isothiocyanate (FITC); rhodamine and rhodamine derivatives; Texas Red; phycoerythrin; allophycocyanin; 6-carboxyfluorescein (6-FAM); 2′,7′-dimethoxy-4′,5′-dichloro-6-carboxyfluorescein (JOE); 6-carboxy-X-rhodamine (ROX); 6-carboxy-2′,4′,7′,4,7-hexachlorofluorescein (HEX); 5-carboxyfluorescein (5-FAM); N,N,N′,N′-tetramethyl-6-carboxyrhodamine (TAMRA); sulfonated rhodamine; Cy3; Cy5; etc.

A specific profile of interest is the analysis of T cells. Arrays of MHC monomers, tetramers, peptide-loaded DimerX (BD-Pharmingen), etc. that provide MHC presentation of antigens can be microarrayed for direct, high-throughput diagnosis/analysis of antigen-specific T cells. Peptide-bearing constructs can be printed on a substrate and bound to a T cell sample of interest. Slowly circulating the sample over the printed region (e.g. using a low flow peristaltic pump and a sealed incubation chamber with inlet and outlet, such as the CoverWell™ perfusion chambers from Grace Biolabs) may increase the sensitivity by giving rare populations of antigen-specific T cells more chances to find targets on the surface. Other means to increase the sensitivity may employ a templated chamber to guide the flow along the different antigen-bearing constructs and/or to increase the number of identical spots of each of the constructs, in a direction that is perpendicular to the direction of flow.

Active profiling (AP) and functional binding assays (FBA). In an AP assay, the presence of a given marker is indirectly detected by assaying the fingerprints of its activation. An FBA is a specific type of AP, in which a printed cue (effector probe) actively induces cells to bind to a co-spotted cue (capture probe). In this case, the presence of the receptor involved in the activation is assayed by the induction or enhancement of cell binding. FBA can be used to screen for cues capable of enhancing cell binding to a particular ECM component or CAM; for ECMs and CAMs to which cells can bind following the activation by a specific cue.

Similarly to passive profiling, functional binding assays can be performed in an absolute or a differential manner. However, unlike passive profiling, the capture probe in a functional binding assay is either co-spotted with an additional, effector probe or juxtaposed to an effector probe (e.g. the latter will be present on an adjacent spot). Other examples of active profiling, which do not necessarily involve the induction or enhancement of binding, include any assayable change in one or more cell parameters on spots that contain a given signaling probe, vs. those spots that that do not contain that signaling probe. For example, the presence of a specific growth factor receptor can be inferred from a reproducible increase in cell proliferation only on spots that contain the corresponding growth factor.

It will be understood by those of skill in the art that some capture probes also elicit a cellular response. Even antibodies may be effectively used in the context of an active profiling assay if binding stimulates or blocks a receptor or other marker in a manner that can be detected with another reporter. For example, T cells may be stimulated by co-printed CD3 and CD28, followed by up-regulation of CD69, which can then be detected by immunostaining of cells on combined CD3 and CD28 spots vs. just CD3 or just CD28 spots. In this case, up-regulation of CD69 on the combined spots would indicate the presence of both CD3 and CD28 on the cell surface, even when the level of one of the two markers (say CD28) does not suffice to capture the cells on the corresponding antibody (in which case, the cells would only bind the combined and the CD3 spots, and the CD69 up-regulation would refer only to the combined vs. CD3 spots).

An effector probe can be detected for its ability to enhance the binding of cells to a particular binding probe, and/or for other changes in phenotype. For example, a signaling probe may induce expression of a cell surface marker. While the starting cell population will be unable to bind to the counterpart binding probe, cells responding to the signaling probe will bind.

Results of active profiling assays can be read out as the absolute or differential scores. Readouts of interest include calcium flux following stimulation, changes in expression of markers including reporter genes, and cell surface receptors, changes in BrdU incorporation corresponding to changes in proliferation rates, pulses of voltage sensitive dyes following the induction of electrical activity, changes in cell motility, etc.

One embodiment of active profiling assays is screening for activity of drug candidates, by printing with or without a capture probe. Candidate agents include agents that act inside the cells, and on the cell surface, as described above. To improve the interactions with cells, candidate agents may be printed onto a film-coated slide or in a 3D gel. Sustained release of an agent can be achieved by printing a mixture that releases active agents from a polymer gel or by slow hydrolysis of a linker, through which the active agent is connected to the surface.

In some embodiments, the candidate agent is bound to a polypeptide carrier, which may be a capture probe, a receptor that specifically interacts with the agent, and the like. For example, steroid compounds may be presented in conjunction with their appropriate carrier protein, e.g. retinol binding protein, corticosteroid binding protein, thyroxin binding protein, etc.

Included in the candidate agents that may be screened are arrays of peptide libraries. Peptides, which may provide effector and/or capture functions, are tested by exposing cells to an arrayed library, which may be random sequences, shuffled sequences, known sequences that are randomly mutated, etc. Reactive side chains may be capped prior to the immobilization and uncapped just before applying the cells. The peptides can be bound to the substrate directly, or via a linker attached to one end, bound to a carrier protein, etc. The peptides may be synthesized directly onto the substrate, (see, for example, U.S. Pat. No. 5,143,854).

Migration assays. An aspect of active profiling is a migration assay. In a migration assay putative chemo-attractant cues are printed next to and/or together with a capture molecule. The migration of cells is detected, and compounds scored for their ability to direct such migration. In one embodiment, the directed movement of cells toward nearby chemokine-containing spots, e.g. SDF-1, and/or up a gradient of a chemokine. Such a gradient can be set by increasing the chemokine concentration from spot to spot and/or printing on a substrate that supports the diffusion of printed proteins (e.g. a commercially available collagen gel such as “VITROGEN 100”). The chemokine may or may not be printed with a capture moiety. Also, the cells can either be specifically immobilized with a binding probe, or could be grown un-patterned within a 3 dimensional gel, that is later printed with chemokine fields.

Another embodiment for high-throughput migration assays places cells of interest on top of two ECM gel layers, where the top layer is very thin, having a thickness of from about 0.05 to about 0.2 mm, and the bottom layer is thicker, having a thickness of from about 3 mm to about 5 mm. A 3D array of candidate chemoattractants is printed on one of the layers, and the migration of cells across the layers in response to diffusing chemoattractants is scored. Where there is upward diffusion of chemoattractants would stimulate downward cell migration. Down-migrating cells would cross over to the bottom layer, and the chemotactic activity of each factor is scored by the number of crossing over cells in the portion corresponding to that factor. Alternatively, the cells are placed cells below an empty thin layer, which in turn lies below the printed thick layer. The thin layer may also be replaced with any other layer that can be traversed by cells that are responding to chemotactic agents (for example, transwell filters that are commonly used in standard migration assays).

Migration and spreading of cells out of the printed regions are. associated with secretion of ECM components that can be required for attachment and migration. Such secretions can be locally analyzed by standard immuno-staining against specific components that may be secreted.

Cell-cell interaction assays. The ability to specifically capture any type of cells onto defined locations and to form patterned surfaces with feature sizes on the order of one or few cell diameters, can be used to juxtapose two or more different cell types, and study their mutual interactions. Different cells can be immobilized within the same spots by printing a common binding probe or co-printing of two or more cell-type specific binding probes. Alternatively the cells can be immobilized separate, nearby spots using cell-type-specific binding probes. If cell-type-specific capture molecules are not known, the cells can be screened in an absolute or differential profiling experiment to determine suitable binding partners.

In order to obtain juxtaposition of distinct cell types on nearby spots, those populations may need to be segregated, such that each spot will include only one cell type. This can be achieved by performing an initial screen of cell-type-specific binding partners to screen for binding probes that segregate these populations (as judged by morphology, marker profile, or any other suitable method). For example, one can segregate a mixture of neural and vascular progenitors by exposing the cells to an antibody array that includes a set of antibodies against putatively unique endothelial markers and another set for neuronal/glial-specific markers. The slide can then be simultaneously stained with at least one antibody from each set, to find binding probes within these sets that provide optimal segregation. These binding probes are then be printed at the desired pattern on another array, and thus used for simultaneous segregation and juxtaposition of neural and endothelial progenitors. Subsequently, the cells can be co-cultured and the juxtaposed cells can be compared to non-juxtaposed cells that were captured and cultured on the same slide. An alternative approach can print different cell types onto nearby spots using a non-contact printing technology.

Two staged cell interactions. Another specific profile of interest, which may be a passive or an active profile, involves delayed cell patterning. In such cases, they cells do not immediately bind to the binding probes, but when maintained in culture for a period of time, e.g. about 12 hours, 24 hours, or over several days, over time will come to bind to the spots. This may be due to changes in the cell phenotype, e.g. in response to local environment, or due to low level binding. Delayed patterning can also occur either on a non-specifically reactive surface or within ECM gel arrays, wherein the cells are cultured in the gel prior to the printing, and/or when cells are dispensed in the vicinity of already printed cues.

Cell-fate manipulation. In one aspect, active profiling detects the effects of an agent on cell differentiation. Cells suitable for such assays include a variety of progenitor and stem cells. Stem cells of interest include hematopoietic stem cells and progenitor cells derived therefrom (U.S. Pat. No. 5,061,620); neural crest stem cells (see Morrison et al. (1999) Cell 96:737-749); embryonic stem (ES) cells; mesenchymal stem cells; mesodermal stem cells; etc. Other hematopoietic “progenitor” cells of interest include cells dedicated to lymphoid lineages, e.g. immature T cell and B cell populations. Progenitor cells have also been defined for liver, neural cells, pancreatic cells, etc. Profiling may screen molecules that can direct differentiation, de-differentiation and trans-differentiation events. In particular, the control over ES cell differentiation is especially important for both regenerative medicine and for understanding the very early stages of mammalian development. A common theme in development is the influence of local morphogens on cell-fate decisions. The methods of the invention provides means of rigorously and systematically exploring the actions of concentrated purified morphogens (e.g. Notch, BMP-4, Wnt-1, bFGF, Shh, their modified forms, other members of their families, etc) by constructing local (discrete or continuous) gradients and fields thereof, to which the cells of interest can be exposed and then profiled. It can also be used to examine the effects of their immobilization, association with matrix components or mixtures, or with one another.

Local effects can be obtained by immobilized (membrane-bound and/or ECM-bound) signaling probes; high local concentrations of secreted cues from adjacent cells; differential cell response to different concentrations of a signaling probe; to combinations of signaling probes; and the like. The cell microarray platform offers a unique opportunity to mimic those scenarios in a very high-throughput manner. Thus, for example, fields of immobilized or diffused morphogens, e.g. Shh, FGFs, Wnts, Notch, TGFs etc., and many other cytokines/growth factors/hormones can be deposited at arbitrary combinations and concentrations, usually in combination with a binding probe, e.g. CAM, ECM component, etc. Alternatively, the stem or progenitor cells may be embedded in a three-dimensional matrix (described in more detail below), where the use of a binding probe is not necessary.

Additional factors that can be deposited on the microarray are conditioned mediums, and cell fragments. Undifferentiated ES cells can be cultured on such arrays and can be screened for spot (bound) and medium (unbound) conditions required for the appearance of a desired differentiation phenotype. The latter can be detected as a morphological feature, e.g. the appearance of elaborate neuronal processes in the case of neuronal differentiation, cell contractions for myocytes, etc.; by a lineage-controlled reporter gene; staining with a set of lineage restricted markers; and any of the standard readouts that are used to phenotype cultured cells.

Both the morphological and lineage-controlled reporter gene readouts can be continuously monitored in real time and/or recorded time-lapse using commercially available systems for live cell recording that have scanning capabilities and are equipped with a proper environment control system (e.g. the Axon Instruments “ImageXress” system).

In addition to the above described formats, the assays of the invention may use three dimensional gels, e.g. an ECM gel such as “VITROGEN 100” collagen gel, (Cohesion Technologies, Inc). The probes may be printed on the gel within which cells are pre-embedded; signaling probes may be printed together with binding probes, or followed by exposure to the cells and washout of non-attached cells. Alternatively the cells may be printed together with signaling probes (provided that the gel is properly hydrated).

Printing onto gels can be performed with a non-contact micro-dispensing system, e.g. Packard Bioscience “Biochip Arrayer”. Such systems utilize a non-violent dispensing mechanism (contraction of piezzo-electric sleeve). Tips with a relatively wide open, e.g. at least about 75 μm, that provide for drops of a volume of greater than 300 nl. volume of each dispensed drop (0.350 nL), allow for cell deposition along with signaling probes of interest. A positioning camera can allow probes and cells to be locally added at later stages.

The three dimensional array and some film coated slides as substrates for printing allows for diffusion of signaling probes, where the effect of a gradient on a cell can be analyzed. The printed probes diffuse and form potentially important continuous gradients.

ES cells can be applied and washed away from the surface of an un-printed “VITROGEN” collagen gel, or can be cultured within it by mixing them with the neutralized liquid phase of the gel prior to gelation (fibrillogenesis), initiating gelation by raising the temperature from 4° C. to 37° C., and culturing the (solid) gel in a standard ES medium.

The agents utilized in the methods of the invention may be provided in a kit, which kit may further include instructions for use. Such a kit may comprise a printed microarray. The kit may further comprise cells, assay reagents for monitoring changes in cell phenotype, singling probes, and the like.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### EXAMPLE 1

### Preparation of Cell Profiling Array

A cellular microarray was assembled, using different capture, effector, detector and soluble probes, where the capture probes are proteins capable specific binding to molecules present on the cell surface, effector probes can effect the cells phenotype, detector probes allow detection of secreted molecules and soluble probes reflect a feature of the cell. Cells were then incubated on the array to provide for specific binding and spatial distribution of the cells.

Methods

Array preparation: Solutions of probe proteins were prepared: at concentrations ranging from 0.01 μg/μl to 1.0 μg/μl, diluted in PBS buffer without glycerol. The proteins were spotted onto hydrated gel slides (Hydrogel slides).

The HydroGel slides require, in addition, pre-processing to remove the storage agent present in the substrate (as well as to ensure consistent, uniform substrate condition), and post-processing to immobilize the proteins. Pre- and post-processing of the HydroGel slides was performed as described in the HydroGel protocol guide.

The proteins were prepared in a 384-well microtitre plate. The proteins on a single array are the same or different depending on the printing plan. Printing was performed with 8- to 32-tip print head, depending on the desired print area and the number of different samples to print. The typical local density of the printed spots was (3265/cm² (spot to spot distance of 175 μm) and the maximal density is 4444/cm² (150 μm)). The arrays were sealed in an airtight container. They can be stored at 4° C. for short term storage (˜1-2 month) or frozen for longer storage.

The back side of the slides was marked with a diamond scribe or indelible marker to delineate the location of groups of spots. In some cases, printed FITC-, Cy3- or Cy5-conjugated BSA (at 0.2 μg/μl) and positive control spots (to which the cells were known to bind at high numbers), were used as coordinate systems.

### EXAMPLE 2

### Molecular Profiling of Cancer

Biologic samples containing or potentially containing cancer cells are analyzed for their molecular profile for purposes of diagnosis, prognosis and therapeutic options. Such samples were taken from peripheral blood, biopsy samples, tissue culture or any volume of fluid which contain cells. Pre-processing of biologic samples involved one or more of the following: a) direct application to the array surface b) dilution in PBS prior to application to the array, c) centrifugation, followed by resuspension in PBS or media (PBS, RPMI, DMEM, culture media), prior to application to the array, d) removal of red blood cells by ammonium chloride e) isolation of PBMC by Ficol gradient purification f) purification of a particular population of cells by FACS g) enzymatic dissociation of solid tissue usually with collagenase h) mechanical dissociation i) forceful filtering with a pore size greater than a single cell of interest (5-70 uM pore size. In figure, peripheral blood from a patient with leukemia was ficoll purified and resuspended in RPMI containing 5% fetal calf serum prior to application of 1×10⁶ PBMC to the array in 500 ul at 37° C. for 5 minutes. The array was then dip washed briefly in PBS and inspected. In figure , a surgically resected melanoma sample was cut using a surgical blade, and enzymatically dissociated with collagenase for 20′ at 37° C., strained through a 70 um filter, ficol purified, and resuspended in RPMI containing 5% fetal calf serum prior to application of 1×10⁶ PBMC to the array in 500 ul at 37° C. for 20 minutes. The array was then dip washed briefly in PBS and inspected a) and

Arrays were blocked with BSA or serum containing media such as 5%FCS/PBS, or pre-wet with PBS (as noted in figures or tables?). Cells were applied directly to the surface of the microarray at a concentration between 10³/ml and 10¹⁰/ml. Cells were allowed to interact with the array for a period of time, usually from 5′ to 60′. Binding was performed at 4 degrees, room temperature or 37 degrees C. Binding was usually performed at room temperature or 37 degrees. At 4 degrees, incubation time was extended.

After incubation, arrays were rapidly dipped in washing buffer. Washing buffer can be any suitable media, but commonly is PBS, RPMI, or serum containing media. After dipping, the arrays were fixed in paraformaldehyde containing solution (1% paraformaldehyde/PBS for 10′), stained, or kept wet in PBS or suitable culture media. Imaging was performed with cells adherent to the array spots. This allows for correlation between individual cells and microscopic features, molecular and/or functional profile. In some examples, the cells were removed from the array by pipet, aggressive washing in PBS, or a mild detergent such as 1-5% triton X in PBS. Alternatively, cells may be exposed to a condition which leads to cell death, prior to fixation. Dead cells detach from the array due to degradation of molecules accounting for attachment to the array.

In figure, after specific immobilization of cells on the surface of the array, the cells were then exposed to a fluorescent functional marker. In figure , the marker is C12resazurin, which fluoresces in reductive environments. In this case, non malignant cells fluoresce, but the leukemia cells do not. In figure, the marker is a fluorescent deoxyglucose, which accumulates and fluoresces in cells with increased metabolic and glucose consuming activity. In this example, the malignant melanoma cells fluoresce, but the benign cells do not. In figure, after immobilization on the array, cells are fixed, permeabilized and stained with a combination of fluorescent markers, allowing identification of different cell types immobilized on the same array spot. In this case, anti-GFAP and anti-tuj1 differentially label the neuronal and glial cell types. BrdU labels the nuclei of dividing cells.

Molecular profiles may be inspected by eye, or microscopy, or high-throughput microscopic data acquisition. Cell type specific signal may be determined by correlation of microscopically identified cell populations (leukemic cells counter stained with Wright's Giemsa Stain are obvious, and only spots containing these cells are considered part of the molecular profile for that cell population; carcinoma cells are often similarly morphologically distinct; properly counter stained lymphocytes are distinct from monocytes, etc.). Alternatively, counter staining with a labeled antibody or secondary antibody may also allow cell specific signal separation. B cell malignancies are often labeled with aznti-CD20 antibody, which may be directly conjugated to a fluorophore such as phycoethrithrin (PE), FITC, Cy5, Cy3, biotin, HRP or quantum dots. Once labeled, these cells may be examined for specific molecular and functional profiles (e.g. CD10, CD19, CD20, CD23, CD34, CD44, CD99 surface expression, IL-4, IL-10, TGF-β secretion from the labeled B cell malignancy, where as T cells in the same preparation show CD3, CD8, CD34, CD44 surface expression and IL-2, IFN-γ secretion). Alternatively, different cell types can also be differentiated by differences in behavior. Fluorescent dyes such as C12Rezulin, Rhodamine123 or 3NDBG can be added to cells on the array. Development of a fluorescent signal after incubation for 15′@ 37C indicates differences in reduction capacity, mitochondrial voltage and glucose uptake and metabolism respectively. We have used such dyes to label benign but not malignant cells, malignant cells and melanomas and hematologic malignancies respectively across multiple clinical samples.

Automated data acquisition is enabled by the regularity of spot printing. Once an initial spot is identified (whose position does not vary by more than 1 μm to 1 mm from array to array), a regular spot center to spot center offset, column and row number, and known probes at each position allows automated capture of a brightfield image (with or without DIC or phase contrast), and fluorescent channels (such as UV, FITC, PE, CY3, CY5, etc . . . ) in 3 dimensions (as needed, a z-motor objective or stage allows capture of images in multiple z planes around a center which provides 3D reconstruction). Image processing allows automated statistics including cell count in brightfield or different fluorescent channels (if anti-CD20 PE and anti-CD8 FITC were used for counterstaining, then the cell count in the PE channel would correspond to CD20+ B cells/B cell malignancies and the cell count in the FITC channel would correspond to CD8+ T cells), average spot signal intensity, etc . . .

Functional Profiling of Cancer

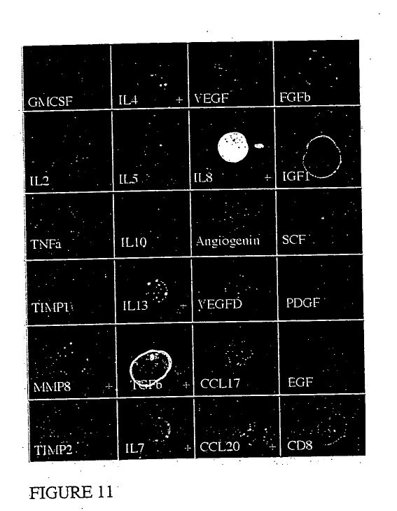

Samples are processed and analyzed as with molecular profiling of cancer, as follows. Arrays used in the functional profiling assays used the capture probes CD20, C44, and CD14. A detector probe were unlabeled, and were one of the following: anti-VEGF, anti-VEGFD, anti_MMP8; anti-TIMP1; anti-TIMP2; anti0IL8, anti-angiogenin, anti-FGFB, anti-IGF, anti-SCF, and PDGF receptor, as shown in FIG. 11. Each detector was used at a stack concentration of 2 mg/ml, and mixed with a capture antibody at 0.5 mg/ml in a 1:1 mixture. The final concentration was 1 mg/ml of detector.

The mixture of probes was placed in an 84 well plate for printing, and printed with a non-contact printer on a gel-based surface. After printing the slides were humidified for 24-48 hrs in a humidification chamber. Slides at kept at 4 degrees until use. At time of usage the slides were used as is, or pre-wet in PBS or PBS+5% FCS for 1-5 minutes. Excess water is removed.

Clinical samples of lymphoma cells after Ficoll separation were prepared in deficient RPMI, or PBS. Cells were applied to the surface of the array in about 50-100 μl. Alternatively the cells were applied with a lister slip, and incubated on the array for 10 minutes at room temperature, then dip-washed in PBS. Cells are then incubated, usually at 37 degrees C for 2 or 24 hours

The cells were dipwashed again, and exposed to a developer solution comprising an antibody specific for the factors detected by the detector probes, where the antibodies are non-interfering, i.e. a second anti-VEGF antibody that binds to a non-interfering site from the detector anti-VEGF antibody. Each of the developer antibodies is labeled, usually biotinylated. The developer is applied in 10% FCS in PBS, and allowed to incubate at 20 minutes for room temperature.

The sample is then dipwashed again, and the fluorescent reagent was added at a concentration at 0.5 mg/ml streptavidin PE, diluted in 10% FCS/PBS and allowed to bind at 20′room temperature in the dark. The slide was then dipwashed and visualized. The results are shown in FIG. 11.

Molecular profiles may be inspected by eye, or microscopy, or high-throughput microscopic data acquisition. Cell type specific signal may be determined by correlation of microscopically identified cell populations (leukemic cells counter stained with Wright's Giemsa Stain are obvious, and only spots containing these cells are considered part of the molecular profile for that cell population; carcinoma cells are often similarly morphologically distinct; properly counter stained lymphocytes are distinct from monocytes, etc.). Alternatively, counter staining with a labeled antibody or secondary antibody may also allow cell specific signal separation (see molecular profiling, above).

Functional signals may have distinct patterns, depending if secretion of these factors is focused or diffuse, weak or strong.

Functional profiles generated are not limited to cell array applications, but may be applied to co-spotted beads, and analyzed in a plate or well, or by flow cytometry. Co-spotted wells are another possible application to which cells are added and response analyzed.

Profiling Heart Disease

Blood samples and or artherectomy samples are processed as with cancer samples. Here, removal of neutrophils may not be desired, so RBC lysis or buffy coat preparation rather than Ficoll may be preferred. A known number of cells are then added to an array that includes spots with capture probes which contain lipids or lipoproteins. Incubation for 5′-30′ at room temperature or 37C is usually sufficient for lipid specific cells to interact with and stably bind to these spots. The array is then washed and the number of cells binding to different lipids and lipoproteins may be correlated to specific clinical risk of heart disease and specific differences in cellular interactions with lipids.

In figure, peripheral blood samples from healthy individuals or a patient with acute coronary syndrome were studied. Samples were ficol purified, and resuspended in 5% FCS/RPMI and added to the lipid array for 20′ at room temperature or 37° C. Arrays were then dip washed and imaged.

Alternatively, if functional profiling is enabled, by using an array with lipid or lipoprotein co-spotted with detector probes, cells binding to and interacting with lipid may be further incubated at 37C for 30′-48+ hours. The arrays are again dip washed. If biotin or HRP conjugated developer are added, SA conjugated to a fluorophores or HRP substrate are then added. However, if cell surface background is present, the cells may either be removed (as above), or the array may be further incubated at 37C for 30′-12+ hours, which allows surface bound developer to be internalized prior to final development of the signal.

Association of particular numbers of cells, cell types or secretion of specific factors in response to association with specifc lipids and lipoproteins may be correlated with clinical outcome, risk of heart disease, and therapeutic response profiles.

Cellular lipid response profiles are not limited to cell array applications, but may be applied to lipid coated beads, and analyzed in a plate or well, or by flow cytometry. Lipid coated wells are another possible application to which cells are added and response analyzed.

Profiling Infectious Disease

Blood, urine, or drinking water that contain, or might contain microbial agents such as virus, bacteria, parasites, fungi, molds is processed to remove red blood cells as above. The sample is then added to the surface of the microarray, where it is captured on different array spots based upon the molecules expressed on the micro organism's surface. If the organism is not visible by simple microscopic inspection, a fluorescent secondary antibody or fluorescent lipophilic marker may be added to highlight the microbes. Secreted factors, such as endotoxins, may also be profiled by co-spotting with a secreted factor capture molecule (detector probe) and developed by a fluorescent developer.

Multiparameter Profiling

The cellular microarray is capable of analyzing a cellular sample for separate features/cell surface molecules. However, if the investigator wishes to analyze several molecules expressed on the same cells, a modified approach is required. For example, if one wishes to know whether a population of cells that is positive for 4 separate molecules is present, such as CD3, CD8, CD45RO, and CD69 on the same cells, this can be performed by counterstaining with 3 of the 4 factors, on the cells immobilized to the 4^{th} factors spot. Hence, cells binding to the CD3 spot can be stained with anti-CD8 FITC, anti-CD45RO PE and anti-CD69 Cy5. In the case of increasing number of factors that are required to be stained for, use of a system such as antibodies conjugated to quantum dots is recommended.

Environmental Profiling

When a pathologic specimen is excised from a patient, it represents a temporal snapshot of molecular events occurring at a given moment. However, events which trigger progression of the disease course, such as metastasis of cancer cells, may have already occurred, or have yet to occur. However, understanding how these cells would behave given particular situations may tell us a great deal regarding what the cells are capable of.

To characterize tumors on an individual basis is possible by presenting the tumor cells to specific scenarios, and seeing how they react. This is achievable on the cellular microarray. Excised tumors are exposed to a functional profiling array, and captured on specific spots. Unbound cells are washed off. The cellular microarray is then exposed to specific environmental stimuli, some of which are listed below: a) hypoxia; cells are incubated at 37 degrees C at lowered oxygen levels to simulate hypoxia in the tumor microenvironment which can trigger expression of metastatic/angiogenic factors. Oxygen is commonly lowered to 7%, 5%, 2%, 1% or 0% b) serum starvation; cells are incubated in a media with either lowered or lacking serum stimulation c) Glucose; cells are incubated in a media containing a decreased or no glucose d) pH; cells are incubated in a media at a higher or lower pH e) Factors; tumor cells can react to specific factors present in their environment. These include chemokines which can lead to homing of cancer cells to distant locations in the body, immunity related factors such as interferon, which can lead to expression of molecules or factors which protect the cancer cells. Molecular profiling of cells prior to and after exposure of cancer cells to such environmental cues is also of interest, and readily performed by adding the cells pre- and post-exposure to these environmental cues to a molecular profiling array.

In figure, colon cancer cells were immobilized on a functional profiling array (the capture antibody in anti-CD44) and incubated at 37° C. at 21% Oxygen, 5% Oxygen and 0% Oxygen for 24 hours. In figure, melanoma cells were immobilized on a functional profiling array (the capture antibody in anti-CD44) and incubated at 37° C. in serum free, glucose free media (PBS) at at 21% Oxygen, 5% Oxygen and 0% Oxygen for 24 hours. At 24 hours, both experiments were developed as detailed above (under functional profiling of cancer)

Gel-Based Elispot

In some instances, specific capture of cells prior to analysis of secreted is not desired. In such cases, the platform presented here still provides advantages over traditional Elispot assays on plastic or glass. These advantages include a 3-D matrix for high protein loading capacity, increased space for secreted factor capture and high resolution. Hence, the capture probe can be omitted in such situations and cells added to a gel matrix coated with detector and/or effector probes in either an array or well based format. Secreted factors are then captured and developed.

Gene Expression Profiling of Enriched Cell Populations

Cells may be profiled as indicated above. However, if gene expression analysis is desired, this can be performed on cells that have been selected or enriched by specific immobilization on the array. Thus, if a clinical sample derived from a breast cancer biopsy is analyzed, it would contain genes from normal epithelial tissue, endothelial cells, fibroblasts, and breast cancers, some of which may be in different states, or present differences in biology. By first applying the sample to the cellular microarray, a specific population of those cells may be analyzed for their gene expression profile. Thus, one could select only those breast cancer cells that expressed 4+ her2neu overexpression for gene expression analysis.

Microscopic Analysis of Enriched Cell Populations

Once cells have been specifically captured to a cellular microarray, microscopic analysis of cellular appearance can be useful. This can be further assisted by application of a pathologic stains such as H&E, DiffQuick, Wright's Giemsa, Trypan Blue or other similar stains. Morphologic appearance can be performed by light, and/or fluorescence microscopy, confocal microscopy, or electron microscopy.

Array Geometry

Not all cellular microarray applications are best performed on a flat surface. Cellular array analysis may also be performed on different surface geometries, such as on the inner surface of a fluid collection or vacutainer tube, or within a capillary. While a fluid collection tube allows convienient ease of use, a capillary allows cells to be drawn across the cell array surface.

What is claimed is:

1. A cell profiling composition comprising:
a high specificity substrate with which is stably associated a pattern of spots of probes; and
cells specifically bound to said probes.

2. The cell profiling composition of claim 1, wherein the non-specific binding of cells to said high specificity substrate is less than about 100 cells/cm².

3. The cell profiling composition of claim 2, wherein said high specificity substrate is a hydrogel.

4. The cell profiling composition of claim 3, wherein said hydrogel comprises hydrophilic components that weakly repulse cells.

5. The cell profiling composition of claim 4, wherein said hydrogel comprises a polymerized mixture of acrylamide, and hydrophilic acrylates.

6. The cell profiling composition of claim 5, wherein said probes are dispensed on said substrate with a non-contact printer.

7. The cell profiling composition of claim 5, wherein said spots comprise a plurality of concentrations of at least one said probe.

8. The cell profiling composition of claim 5, wherein said spots comprise a plurality of probes.

9. The cell profiling composition of claim 8, wherein said plurality of probes comprises effector probes and capture probes.

10. The cell profiling composition of claim 8, wherein said plurality of probes comprises detector probes and capture probes.

11. The cell profiling composition of claim 8, wherein said cells are exposed to soluble detector probes.

12. The cell profiling composition of claim 8, wherein said cells are exposed to soluble effector probes.

13. The cell profiling composition of claim 8, wherein said probes comprise polypeptides.

14. The cell profiling composition of claim 8, wherein said probes comprise lipids.

15. The cell profiling composition of claim 8, wherein said probes comprise carbohydrates.

16. A method of profiling cells, the method comprising:
contacting a population of cells with a microarray, wherein said microarray comprises a high specificity substrate with which is stably associated a pattern of spots of probes; and
determining the effect of said probes on said cells in a site specific analysis.

17. The method according to claim 16, wherein said site specific analysis comprises determining a change in the phenotype of cells bound to said microarray.

18. The method according to claim 17, wherein said population of cells is a heterogeneous population.

19. method for identifying physiologically active cellular mechanisms, signaling or growth factor receptors and/or sensitivity to targeted therapy.
